(19) United States
(12) Patent Application Publication
(10) Pub. No.: US 2015/0351608 A1
(43) Pub. Date: Dec. 10, 2015

(54) METHOD AND DEVICE FOR EVALUATING A COLONOSCOPY PROCEDURE

(71) Applicant: OHIO UNIVERSITY, Athens, OH (US)

(72) Inventor: JungHun CHOI, Athens, OH (US)

(73) Assignee: Ohio University, Athens, OH (US)

(21) Appl. No.: 14/759,236

(22) PCT Filed: Jan. 8, 2014
(86) PCT No.: PCT/US14/10657

§ 371 Date: Jul. 6, 2015

Related U.S. Application Data
(60) Provisional application No. 61/750,961, filed on Jan. 10, 2013.

(51) Int. Cl. A61B 1/00; A61B 1/31; A61B 1/005

(52) U.S. Cl. CPC A61B 1/00055; A61B 1/005; A61B 1/31; A61B 1/00004; A61B 1/00045

(57) ABSTRACT

A method and device for evaluating a colonoscopy procedure includes an endoscope (12) having a distal portion (22) and a proximal portion (24). A plurality of sensors (28) are positioned along the endoscope (12) and operatively connected to a processing system (14). The plurality of sensors (28) and processing system (14) detect a first bending of the distal portion (22) and a second bending of the proximal portion (24). The first and second bending are compared by the processing system (14) for estimating the formation of the partial loop in order to predict the formation of a full loop during the colonoscopy procedure.

### CROSS-REFERENCE TO RELATED APPLICATION

This application claims the priority of Application Ser. No. 61/750,961 filed Jan. 10, 2013 (pending), the disclosure of which is hereby incorporated by reference herein.

### TECHNICAL FIELD

The present invention relates generally to a method and device for use during a colonoscopy procedure, and more particularly, to a method and device for evaluating endoscopic loop formation during the colonoscopy procedure.

### BACKGROUND

Colonoscopy is a common and effective procedure for screening colorectal diseases of the lower gastrointestinal tract. Medical doctors generally recommend all patients over the age of fifty to undergo the procedure with relative frequency to ensure early detection and treatment of colorectal diseases, such as colon cancer. However, the physical discomfort and related psychological stigma of the procedure may create a barrier to patients unwilling to accept such discomfort for the sake of preventative detection. For this reason, minimizing discomfort related to pressure, pain, or tearing within the gastrointestinal tract is important for improving a patient's experience and safety to encourage routine colonoscopy procedures.

During the colonoscopy procedure, an endoscope, such as a colonoscope, is inserted into a colon, through the rectum, of a patient for viewing the lower gastrointestinal tract. As the endoscope is forced along the colon, the endoscope may kink, bend, or otherwise loop, which hinders the advancement of a distal tip of the endoscope. Such endoscopic looping is considered one of the primary challenges to successfully completing a colonoscopy procedure. The loop tends to create additional pressure within the gastrointestinal tract and necessitates an operator to apply additional force in order to advance the endoscope through the colon. Unfortunately, the additional force tends to increase discomfort, pain, and may even tear the mucosa of the colon itself by stretching the mesentery attached to descending and ascending colons of the lower gastrointestinal tract.

Various devices and methods are known to reduce endoscopic looping. These devices and methods generally include both active and passive looping prevention. On one hand, passive looping prevention generally includes extensive education of anatomy and practice using colonoscopy simulators to develop skills for preventing loop formation. In addition, water injection, abdominal pressure, and patient position may passively help to prevent loop formation. On the other hand, active prevention includes the addition of a device to the endoscope that actively deters the formation of the loop and may include double balloons, variable stiffness tubes, SHAPELOCK®, and/or overtubes. Such active prevention serves to guide the endoscope along the colon in order to minimize the likelihood of forming the endoscopic loop; however, it fails to detect a formed loop. Thus, active prevention may also include visualization devices that detect a formed loop. The visualization device may include CT colonography, fluoroscopy, and magnetic endoscopic imaging and generally are directed to creating a visual image of the endoscope within the colon.

While known active and passive looping prevention techniques may be simultaneously employed to decrease the likelihood of endoscopic looping, each tends to suffer drawbacks affecting patient comfort. First, each of the above techniques requires a great deal of time and training to properly prepare the operator for performing the colonoscopy procedure. Second, active looping prevention may help to prevent a loop, but still requires significant capital investment. Many active looping prevention systems even fail to actually detect a formed loop if one should occur. While some active loop prevention may help to visualize the formed endoscopic loop, the skill set necessary for the operator to recognize the loop requires even more time and training to complete. Moreover, even if the operator is sufficiently trained to perform the colonoscopy with the additional visualization device, the operator may be distracted by analyzing both an endoscopic camera and the visualization device. As such, the procedure will require additional time and, in turn, greater patient discomfort to complete.

There is a need for a method and device for use during a colonoscopy procedure, such as evaluating endoscopic loop formation, that addresses present challenges and characteristics such as those discussed above.

### SUMMARY

One exemplary embodiment of a method for evaluating an endoscope during a colonoscopy procedure comprises detecting a first bending of a distal portion of the endoscope and detecting a second bending of a proximal portion of the endoscope. The method also comprises comparing the first bending to the second bending with a processing system. Finally, the method also comprises estimating the formation of a partial loop with the processing system for predicting the formation of a full loop.

In one aspect, the method further comprises determining a ratio between the first bending and the second bending to estimate a percentage of the partial loop formed. The method also includes comparing the percentage of the partial loop formed to a threshold percentage. In another aspect, the method includes providing notice to an operator when the percentage of the partial loop exceeds the threshold percentage.

In another exemplary embodiment, a device for evaluating a colonoscopy procedure comprises an endoscope having a distal portion and a proximal portion. Each of the distal and proximal portions have a plurality of sensors. The plurality of sensors are positioned along the distal and proximal portions and operatively connected to a processing system for detecting bending of the endoscope. In addition, the processing system compares bending detected at the distal portion to the bending detected at the proximal portion for predicting the formation of an endoscopic looping during the colonoscopy procedure.

Various additional objectives, advantages, and features of the invention will be appreciated from a review of the following detailed description of the illustrative embodiments taken in conjunction with the accompanying drawings.

### BRIEF DESCRIPTION OF THE DRAWINGS

The accompanying drawings, which are incorporated in and constitute a part of this specification, illustrate embodiments of the invention and, together with a general description of the invention given above, and the detailed description given below serve to explain the invention.

FIG. 1 is a schematic view of an embodiment of a device for evaluating a colonoscopy procedure.

FIG. 2 is a schematic view of an embodiment of a bending sensor for use with the device shown in FIG. 1.

FIG. 3 is a schematic view of a two-dimensional projection of an exemplary endoscopic alpha loop.

FIG. 4 is a chart of a calibration of the bending sensor shown in FIG. 2.

FIG. 5 is a chart of a transitional effect of the two-dimensional projection of the exemplary endoscopic alpha loop shown in FIG. 3.

FIG. 6 is an embodiment of a display shown in the schematic view of FIG. 1.

FIG. 7A is a schematic view of approximately 25% of an embodiment of an endoscopic alpha loop.

FIG. 7B is a schematic view of approximately 50% of the embodiment of the endoscopic alpha-loop.

FIG. 7C is a schematic view of approximately 75% of the embodiment of the endoscopic alpha-loop.

FIG. 7D is a schematic view of approximately 100% of the embodiment of the endoscopic alpha-loop.

FIG. 7E is a schematic view of approximately 125% of the embodiment of the endoscopic alpha-loop.

FIG. 8A is a schematic view of approximately 0% of an embodiment of an endoscopic N-loop.

FIG. 8B is a schematic view of approximately 25% of the embodiment of the endoscopic N-loop.

FIG. 8C is a schematic view of approximately 50% of the embodiment of the endoscopic N-loop.

FIG. 8D is a schematic view of approximately 75% of the embodiment of the endoscopic N-loop.

FIG. 8E is a schematic view of approximately 100% of the embodiment of the endoscopic N-loop.

FIG. 8F is a schematic view of approximately 125% of the embodiment of the endoscopic N-loop.

FIG. 9A is a schematic view of approximately 50% of an embodiment of an endoscopic U-loop.

FIG. 9B is a schematic view of approximately 75% of the embodiment of the endoscopic U-loop.

FIG. 9C is a schematic view of approximately 100% of the embodiment of the endoscopic U-loop.

FIG. 10A is a schematic view of approximately 50% of an embodiment of an endoscopic gamma-loop.

FIG. 10B is a schematic view of approximately 75% of the embodiment of the endoscopic gamma-loop.

FIG. 10C is a schematic view of approximately 100% of the embodiment of the endoscopic gamma-loop.

FIG. 11A is a flowchart of an embodiment of a first portion of firmware for use with the device shown in FIG. 1.

FIG. 11B is a flowchart of the embodiment of a second portion of firmware for use with the device shown in FIG. 1.

### DETAILED DESCRIPTION

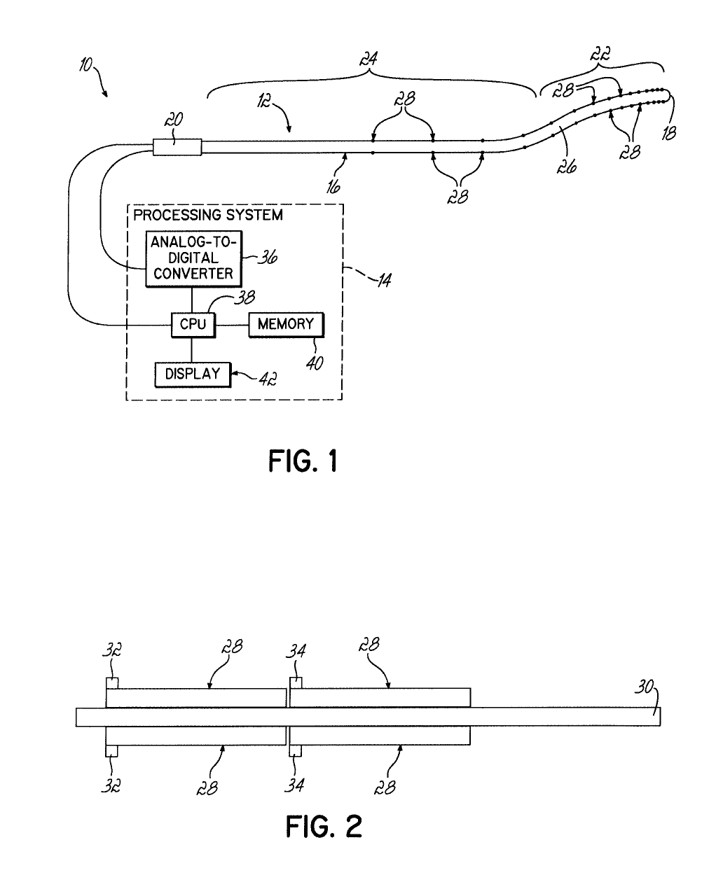

With reference to FIG. 1, an embodiment of a device 10 for evaluating a colonoscopy procedure, particularly with respect to predicting endoscopic looping, includes an endoscope 12 operatively connected to a processing system 14. The endoscope 12 includes a flexible shaft 16 having a distal tip 18. The distal tip 18 may include a camera (not shown), or similar visualization equipment, configured for generating an image within the colon during the colonoscopy procedure. The shaft 16 also includes a handle 20 that may be generally subdivided between the handle 20 and the distal tip 18 into at least two portions relative to the formation of a full or partial loop along the shaft 16. Specifically, the shaft 16 includes at least a distal portion 22 and a proximal portion 24. For reference, the directions along the endoscope 12 generally refer to distal as being toward the distal tip 18 with proximal being relatively toward the handle 20. As such, the exemplary shaft 16 shown in FIG. 1 has a bent portion 26, which marks the subdivision between distal and proximal portions 22, 24. However, should the bent portion 26 be positioned elsewhere along the length of the shaft 16, the distal and proximal portions 22, 24 may vary depending on the particular full or partial loop formed during the colonoscopy. Thus, distal and proximal portions 22, 24 are not intended to be limiting, but, rather, refer generally to portions of the shaft 16 along which any given full or partial loop may form.

A plurality of sensors 28 are positioned along the shaft 16 and operatively connected to the processing system 14 for detecting bending of the shaft 16. According to the exemplary embodiment shown in FIG. 1, each of the plurality of sensors 28 is a uni-directional bend sensor sensitive to detecting the amount of bending at each of the plurality of sensors 28. Specifically, based on the degree of bending, each of the bending sensors 28 changes resistance values for converting to voltage. The uni-directional bend sensors 28 can predict the radius of curvature of the shaft 16 in one axis. With respect to the exemplary embodiment of FIG. 1, thirty bend sensors 28 were used to detect loop formation along the shaft 16.

With respect to FIG. 1 and FIG. 2, two sets of fifteen bend sensors are placed between a continuous substrate 30 to identify both sides of bending. The total length of the positioned plurality of sensors 28 shown is generally 120 cm long and the thickness from top to bottom sensors is generally 3 mm. The plurality of sensors 28 are positioned generally along 72 percent of the shaft 16 with more of the sensors 28 being located toward the distal tip 18. The total length of the endoscope 12 is 160 cm, but from about 10 cm to 130 cm of the shaft 16 is the operable length for insertion into a patient during the colonoscopy procedure. Generally, it will be difficult to form a loop within generally 30 cm to generally 40 cm of the distal tip 18 or the handle 20 of the endoscope 12. For example, in the case where the length of the shaft 16 is generally 30 cm, the radius of the curvature of any full or partial loop will be generally 5 cm and capable of producing relatively high reaction forces within the patient's colon.

Each of the plurality of sensors 28 is operatively connected to the processing system 14 and includes a first terminal 32 and a second terminal 34. The first terminal 32 is connected to excitation voltage while the second terminal 34 is operatively connected to the processing system 14. Furthermore, the processing system 14 generally includes an analog-to-digital converter 36, a CPU 38, a memory 40, and a display 42. Specifically, the second terminal 34 is electrically connected to the analog-to-digital converter 36, which is operatively connected to the CPU 38. The CPU 38 is also operatively connected to the memory 40 for recalling and executing firmware described below in greater detail. Once the firmware is executed, the CPU 38 is operatively connected to the display 42 for visualizing and/or notifying the operator of the results executed by the firmware.

As described above, FIG. 1 and FIG. 2 of the device 10 and the plurality of sensors 28 are schematic. Thus, the invention described herein is not intended to be limited to the specific embodiments shown. For example, any endoscope 12 for use in a colonoscopy procedure may be so used. Similarly, the plurality of sensors 28 and processing system 14 are not intended to be limited to use with resistive bend sensors. It will be appreciated that any sensors or method of sensing the relative position of the endoscope 12 during the colonoscopy procedure may be used according to the principles described below for predicting the formation of endoscopic looping. Thus, while the exemplary plurality of sensors 28 directly sense bending, bending may also be indirectly detected via other known methods of sensing the position of the endoscope 12. In this way, the term bending generally refers to full and/or partial loop formation and may be detected directly or indirectly via any known method.

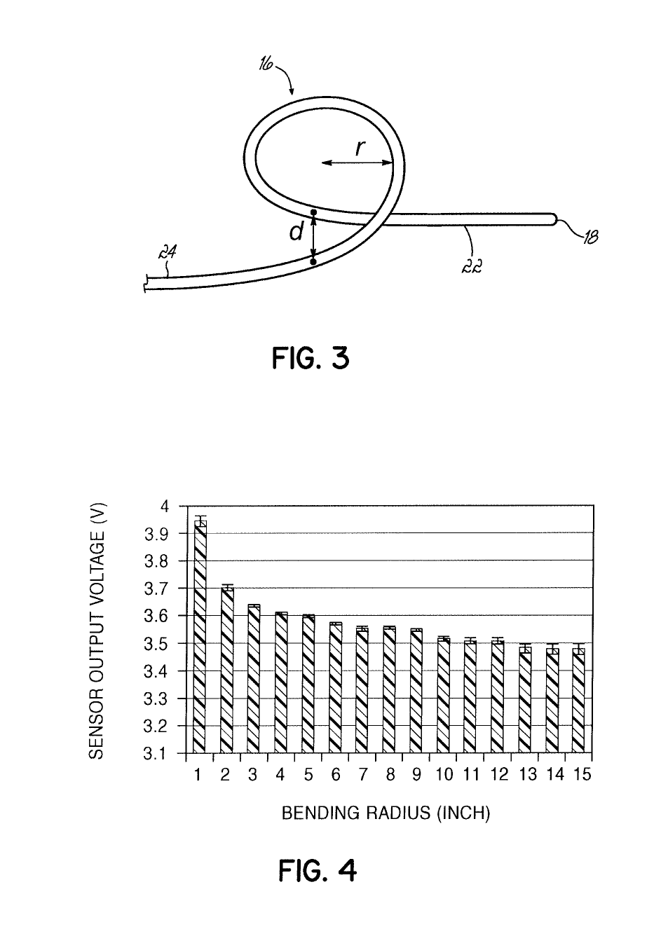

With respect to the exemplary plurality of sensors 28 shown in FIGS. 1 and 2, FIG. 3 depicts the shaft 16 shown in an exemplary alpha-loop formation where “d” represents a three-dimensional loop parameter and “r” represents a bend radius. Generally, when d is approximately zero, the loop is ideally acting on a two-dimensional plane. However, due to the diameter of the shaft 16, approximately 12 mm, d cannot be zero. Still, the loop can be treated on the two-dimensional plane given the relatively large radius of curvature of the shaft 16. As the variable d increases, torsion is applied to the plurality of sensors 28 and increases the values output from the plurality of sensors 28 to the analog-to-digital converter 36 shown in FIG. 1.

FIG. 4 shows the calibration results of one of the bend sensors 28 without torsion, e.g. where d is zero, with respect to FIG. 1. As bending radius, r, increases, the output voltage decreases. Thus, the possible minimum bend radius begins at generally 1 inch, and the maximum bending radius will generally not exceed 10 inches due to the length limitations of the exemplary shaft 16.

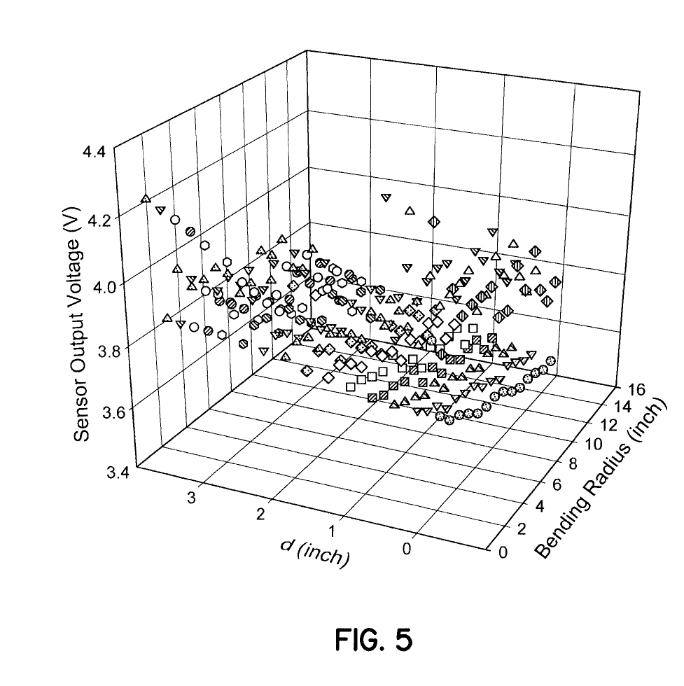

FIG. 5 shows the relation of the output voltage between the two-dimensional loop and the three-dimensional loop. When the variable d increases, the loop opens with a small change of bending radius, and the output voltage from the bend sensor is augmented. Since the maximum value of the variable d does not exceed generally more than 4 inches, a three-dimensional loop can be projected and assumed to be a simple two-dimensional loop. Thus, for the exemplary embodiment of the device of FIG. 1, an N-loop can be detected when the shaft 16 of the endoscope 12 forms two magnitude values of hemi-circles. The values may be the same or different. The variable d is applied to the cases of N-loops or reversed N-loop, and the detection logic is the same as an alpha-loop or a reversed alpha-loop.

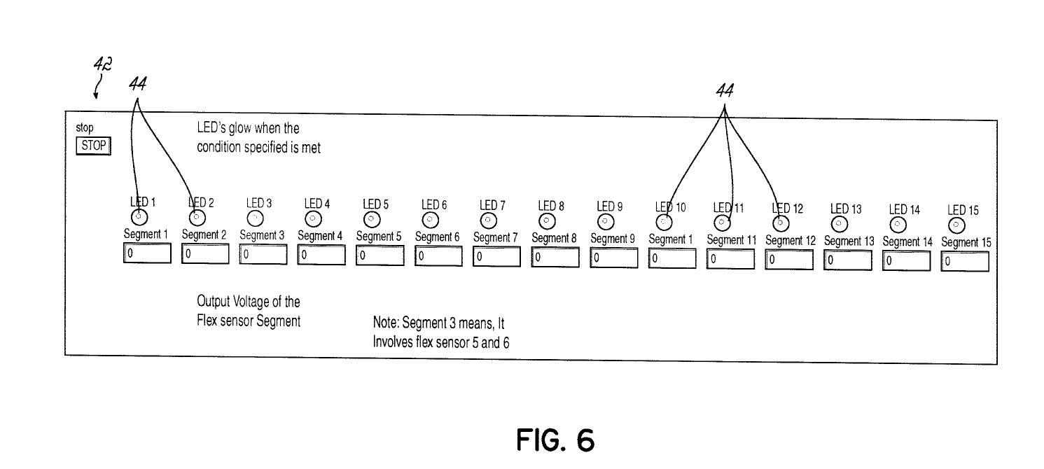

FIG. 6 depicts the exemplary display 42 visualizing the executed firmware described below for the plurality of bending sensors 28 of FIG. 1. The plurality of bending sensors 28 are grouped with the firmware continuously monitoring the bending of each bending sensor 28. Among the groups, consecutive bend sensors 28 values are summed and visualized. In addition, each loop programmed into the memory 40 may visualize a threshold value on the display 42. Generally, when the summation of the consecutive bend sensors 28 exceeds the threshold value for that loop, a virtual LED 44 will indicate the formation of the loop on the display 42.

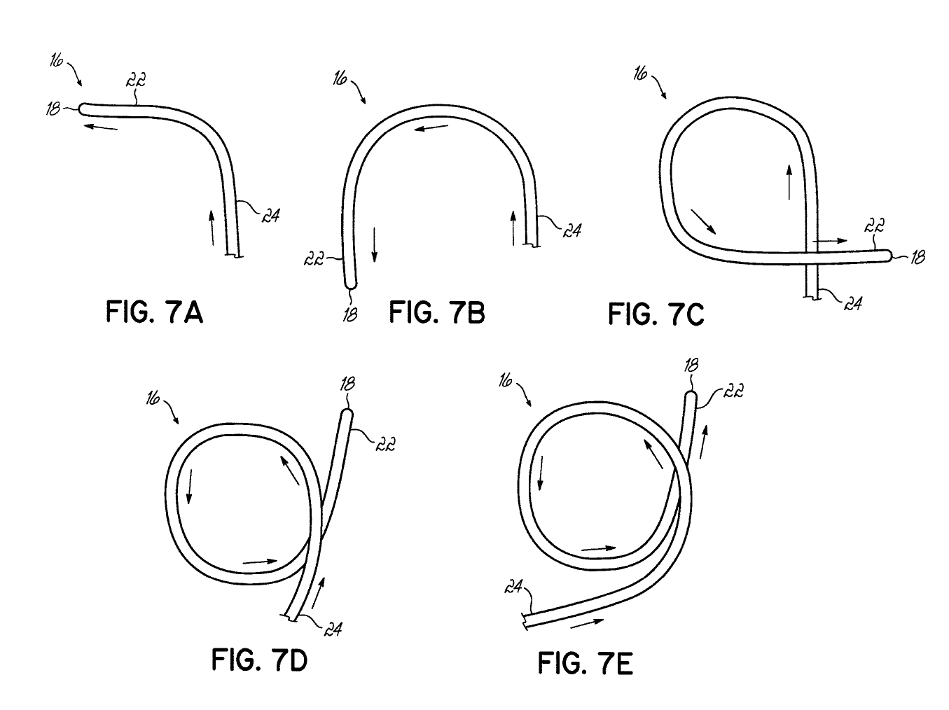

FIGS. 7A-10C show common, exemplary endoscopic loop formations that may be programmed into the memory 40 for comparison to detected bending being continuously monitored by the CPU 38 shown in FIG. 1. FIGS. 7A, 7B, 7C, 7D, and 7E show distal and proximal portions 22, 24 of the shaft 16 forming from a partial alpha-loop into a full alpha-loop. Specifically, FIG. 7A depicts 25% of the alpha-loop, FIG. 7B depicts 50% of the alpha-loop, FIG. 7C depicts 75% of the alpha-loop, FIG. 7D depicts 100% of the alpha-loop, and FIG. 7E depicts 125% of the alpha-loop. As such, the following Table 1 notes the relative angles between the distal and proximal portions 22, 24 and general orientation of the shaft 16.

TABLE 1
Alpha-Loop

| | | | | | |
|---|---|---|---|---|---|
| Loop | 25 | 50 | 75 | 100 | 125 |
| Percentage | | | | | |
| (%) | | | | | |
| Angle | 90 | 180 | 270 | 360 | 450 |
| (Degree) | | | | | |
| Description | 90 is an | The shaft | The | The | The angle |
| of | angle | forms a U | shaft is | direction of | of each |
| General | of a | shape. | crossed. | distal and | distal and |
| Orientation | bending | | | proximal | proximal |
| | shaft. | | | portions of | portion of |
| | | | | the shaft | the shaft |
| | | | | are | becomes |
| | | | | opposite. | 450 (or 90) |
| | | | | | degrees. |

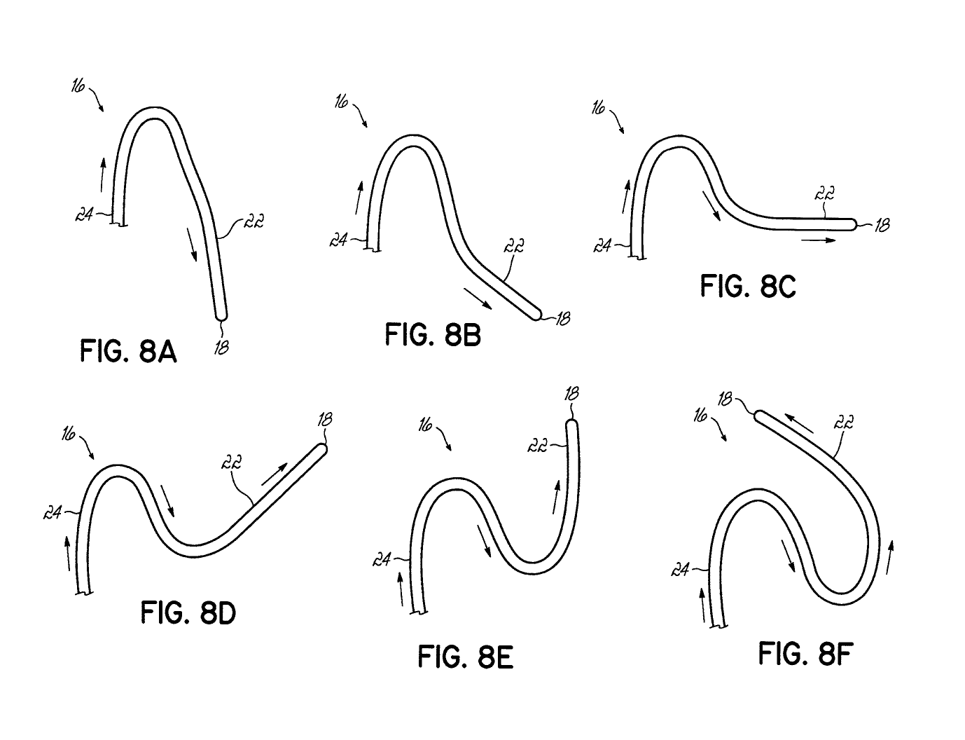

FIGS. 8A, 8B, 8C, 8D, and 8E show distal and proximal portions 22, 24 of the shaft 16 forming from a partial N-loop into a full N-loop. Specifically, FIG. 8A depicts 0% of the N-loop, FIG. 8B depicts 25% of the N-loop, FIG. 8C depicts 50% of the N-loop, FIG. 8D depicts 75% of the N-loop, FIG. 8E depicts 100% of the N-loop, and FIG. 8F depicts 125% of the N-loop. As such, the following Table 2 notes the relative angles between the distal and proximal portions 22, 24 and general orientation of the shaft 16.

TABLE 2
N-Loop

| | | | | | | |
|---|---|---|---|---|---|---|
| Loop Percentage (%) | 0 | 25 | 50 | 75 | 100 | 125 |
| Angle (Degree) | 0 | 45 | 90 | 135 | 180 | 225 |
| Description | The shaft is | Distal portion | Distal portion | Distal portion | Distal and | Distal portion |
| of General | a reversed | of the shaft | of the shaft | of the shaft in- | proximal por- | of the shaft |
| Orientation | U shape. | forms 45 de- | becomes | creases its angle | tions of shaft | bends 45 degree |
| | | grees from | horizontal. | by 45 degree from | are parallel | to the left from |
| | | the vertical. | | the horizontal. | to each other. | the vertical. |

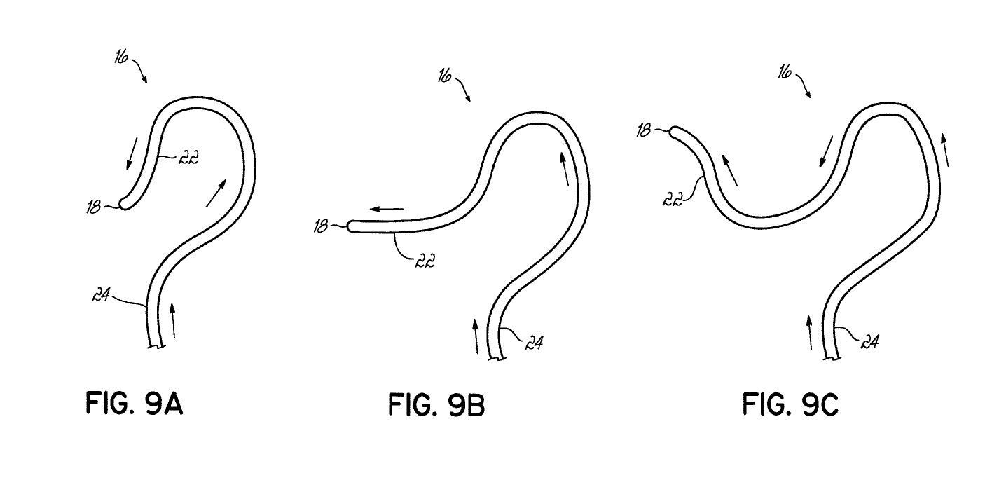

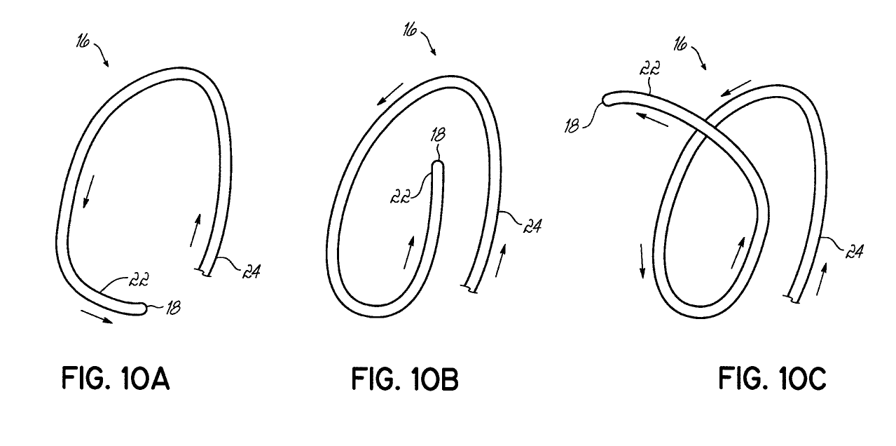

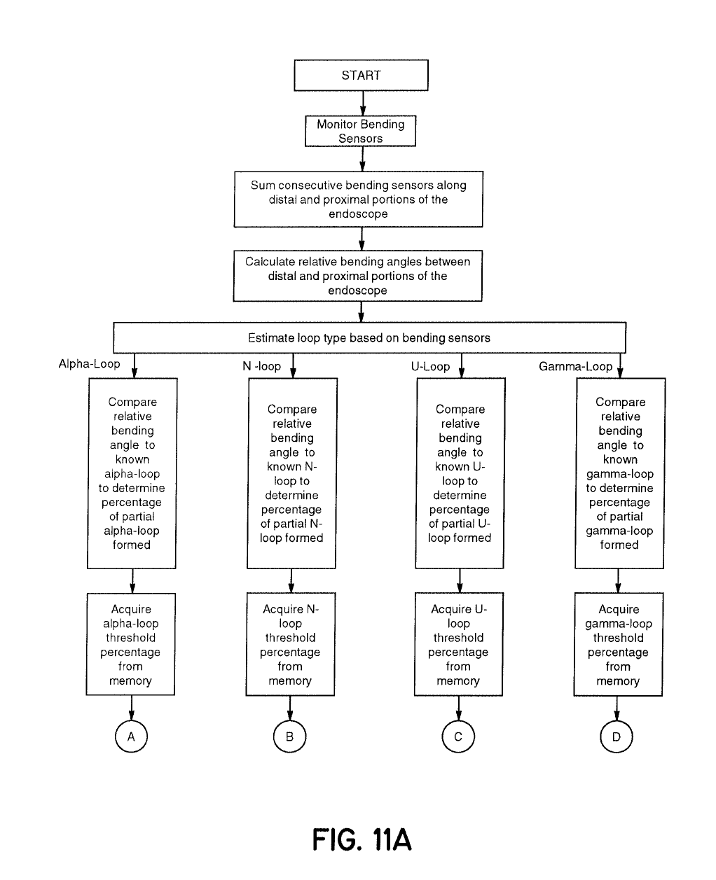

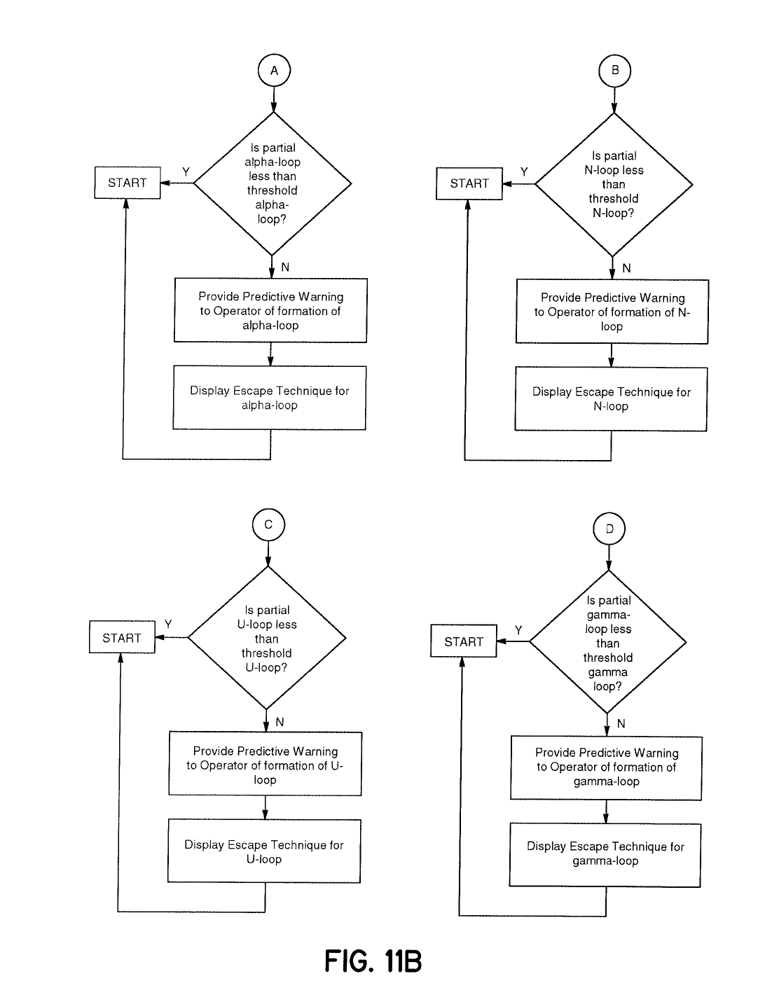

FIGS. 9A, 9B, and 9C show distal and proximal portions 22, 24 of the shaft 16 forming from a partial U-loop into a full U-loop. Specifically, FIG. 9A depicts 50% of the U-loop, FIG. 9B depicts 75% of the U-loop, and FIG. 9C depicts 100% of the U-loop. In addition, FIGS. 10A, 10B, and 10C show distal and proximal portions 22, 24 of the shaft 16 forming from a partial gamma-loop into a full gamma-loop. Specifically, FIG. 10A depicts 50% of the gamma-loop, FIG. 10B depicts 75% of the gamma-loop, and FIG. 100 depicts 100% of the gamma-loop. It will be appreciated that respective angles between distal and proximal portions may be similarly calculated for U-loops and gamma-loops as those calculated for the exemplary embodiments of the alpha-loop and N-loop described above.

The memory 40 of FIG. 1 is programmed with the exemplary alpha-loop, N-loop, U-loop, and gamma-loop formations shown in FIGS. 7A-10C along the distal and proximal portions 22, 24. In addition, the memory 40 is programmed with threshold amounts, or percentages, that represent the threshold values for each of the above loop formations. According to the exemplary embodiment of the programmed memory 40, the alpha-loop threshold value is 60%, the N-loop threshold value is 60%, the U-loop threshold value is 50%, and the gamma-loop threshold value is 50%. However, it will be appreciated that other loop formations and related loop threshold values, not disclosed herein, may also be programmed into the memory 40 by the operator or another programmer. Thus, the invention described herein is not intended to be limited to the loops shown in FIGS. 7A-10C.

With respect to FIG. 1 and FIGS. 11A and 11B, during the colonoscopy procedure, the endoscope 12 is advanced through the colon of the patient while the firmware programmed into the processing system 14 continuously monitors the plurality of uni-directional bending sensors 28 positioned along the shaft 16. Each of the bending sensors 28 produces a value indicative of the amount of bending occurring at each sensor 28. The CPU 38 sums consecutively placed bending sensors for identifying bending occurring along distal and proximal portions 22, 24 of the shaft 16. Then, the CPU 38 calculates the relative bending angles between the distal and proximal portions 22, 24. The CPU 38 stores the relative bending angle and the values of the summations identified along the distal and proximal portions 22, 24 in the memory 40 and compares the stored angle and values to the programmed loop types shown in FIGS. 7A-10C. Based on this comparison, the firmware estimates, or otherwise predicts, the particular type of loop that may be forming along the shaft 16. According to the exemplary embodiment, the loop type is selected from the alpha-loop, the N-loop, the U-loop, and gamma-loop; however, as mentioned above, other loop types may be programmed into the memory 40 for selection by the CPU 38.

With the loop type selected and acquired from memory 40, the CPU 38 compares the relative bending angle calculated above to the programmed alpha-loop for determining the percentage of the partial loop presenting forming on the shaft 16. For example, such a comparison is similar to that of Table 1 and Table 2 for estimating the percentage of the partial loop formed based upon the calculated bending angle between the distal and proximal portions 22, 24.

Next, the CPU 38 acquires the programmed loop threshold value from the memory 40 and compares the threshold value to the percentage of the partial loop formed. If the percentage of the partial loop formed is less than the threshold value, then the processing system 14 returns to start and continues to monitor the plurality of bending sensors 28. However, if the percentage of the partial loop formed is more than the threshold value, then the processing system 14 is configured to notify the operator of its prediction that endoscopic looping may occur. The processing system 14 may also be operable to provide audible feedback through a speaker (not shown) of the processing system 14, visual feedback via the display 42, and/or haptic feedback via the handle 20. In this way, the operator receives predictive warning of the endoscopic looping automatically and without having to sort through the confusion of analyzing multiple displays throughout the entirety of the colonoscopy procedure.

In addition, the CPU 38, having recognized the partial loop, recalls from the memory 40 an escape technique unique to that particular partial loop. The escape technique is then visualized on the display 42 for the convenience of the operator. Given that the processing system 14 is programmed to display the correct escape technique for the particular partial loop, every colonoscopy, regardless of the operator, may be performed with increased consistency and reliability in order to enhance the patient experience. Specifically, Table 3 below indicates the unique loop type and suggested escape technique for visualizing on the display 42.

TABLE 3
Loop Escape Techniques

| Loop Type | Escape Technique |
|---|---|
| Alpha-loop | Withdraw the shaft and rotate the shaft to clockwise |
| N-Loop | Retract the shaft and make the configuration of the colon |
| | straight |
| U-Loop | Retract the shaft and straight the transverse colon |
| Gamma-loop | Withdraw and twist the shaft to clockwise until the |
| | transverse colon is placed to the conventional position. |
| | Additional abdominal pressure may be required. |

Once the escape technique is displayed to the operator, the firmware directs the processing system 14 to again monitor the sensors and repeat the comparison for predicting endoscopic looping. Thus, the escaped technique is displayed until the operator manipulates the endoscope 12 such that the partial loop formed is below the threshold value.

For example, despite the use of active and passive systems for preventing the formation of a loop, the plurality of bending sensors 28 may detect bending of the shaft 16 that the processing system 14 calculates bending of 135 degrees and predicts the formation of 75% of an alpha loop (see FIG. 7C and Table 1). As such, the processing system 14 then compares the 75% partial alpha-loop to the exemplary alpha-loop threshold value of 60%. Because the 75% partial alpha-loop is above the 60% alpha-loop threshold value, the exemplary processing system 14 provides audible, visual, and haptic notice to the operator and displays the alpha-loop escape technique, i.e., “Withdraw the shaft and rotate the shaft to clockwise.” Finally, the processing system 14 continues to monitor the partial alpha-loop and provide notice to the operator of the predicted full alpha loop until the operator successfully manipulates the distal and proximal portions 22, 24 below the alpha-loop threshold value.

In an exemplary embodiment, the operator may also be able to vary the programmed loop threshold value for earlier or later predictive warning of certain types of loops. As such, the operator may increase the sensitivity of the prediction by lowering the loop threshold value or decrease the sensitivity of the prediction by increasing the loop threshold value. With respect to the alpha-loop example given above, if the operator programmed the loop threshold value at 80%, then the processing system 14 would have been less sensitive and the operator may have continued the procedure until the threshold value reached about 80%. Varying the sensitivity of the loop prediction may be helpful for tailoring the device 10 to particular patient or operator preferences.

While the present invention has been illustrated by the description of one or more embodiments thereof, and while the embodiments have been described in considerable detail, they are not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and method and illustrative examples shown and described. Accordingly, departures may be from such details without departing from the scope of the general inventive concept.

What is claimed is:

1. A method of evaluating an endoscope during a colonoscopy procedure, comprising;
detecting a first bending of a distal portion of the endoscope;
detecting a second bending of a proximal portion of the endoscope;
comparing the first bending to the second bending with a processing system; and
estimating the formation of a partial loop with the processing system for predicting the formation of a full loop.

2. The method of claim 1 wherein the first and second bending is detected with a plurality of sensors, the method further comprising:
continuously monitoring the plurality of sensors for first and second bending.

3. The method of claim 1 further comprising projecting the first and second bending of the endoscope from three-dimensions to two-dimensions.

4. The method of claim 1 further comprising:
calculating the bending angle between the first bending distal portion and the second bending proximal portion.

5. The method of claim 1 further comprising:
determining a ratio between the first bending and the second bending to estimate a percentage of the partial loop formed.

6. The method of claim 5 further comprising:
comparing the percentage of the partial loop formed to a threshold percentage.

7. The method of claim 6 further comprising:
providing notice to an operator when the percentage of the partial loop exceeds the threshold percentage.

8. The method of claim 7 wherein the notice is audible, visual, haptic, or any combination thereof.

9. The method of claim 7 wherein the notice includes escape instructions, the method further comprising:
providing escape instructions to an operator for reducing the percentage of the partial loop below the threshold percentage.

10. The method of claim 5 further comprising:
identifying a predetermined loop type with the processing system.

11. The method of claim 10 wherein the predetermined loop type is selected from the group consisting of an alpha loop, a reversed alpha loop, an N loop, a U loop, and a gamma loop.

12. A device for evaluating a colonoscopy procedure, comprising;
an endoscope having a distal portion and a proximal portion, said distal and proximal portions having a plurality of sensors, said plurality of sensors positioned along said distal and proximal portions and operatively connected to a processing system to detect bending of said endoscope,
wherein said processing system compares bending detected at said distal portion to the bending detected at said proximal portion for predicting the formation of an endoscopic loop during the colonoscopy procedure.

13. The device of claim 12 wherein each of the plurality of sensors is a unidirectional bend sensor.

14. The device of claim 13 wherein each of the plurality of sensors is a resistive bend sensor, said resistive bend sensor having an input terminal and an output terminal, said input terminal operatively connected to an excitation voltage, and said output terminal operatively connected to said processing system.

15. The device of claim 14 wherein said processing system comprises an analog-to-digital converter, said output terminal connected to said analog-to-digital converter.
